(19) United States
(12) Patent Application Publication
(10) Pub. No.: US 2009/0074731 A1
(43) Pub. Date: Mar. 19, 2009

(54) METHOD OF ISOLATION AND USE OF CELLS DERIVED FROM FIRST TRIMESTER UMBILICAL CORD TISSUE

(76) Inventors: Clifford L. LIBRACH, Toronto (CA); Shangmian YIE, Scarborough (CA); Rong XIAO, Scarborough (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, CA

(21) Appl. No.: 12/114,182

(22) Filed: May 2, 2008

Related U.S. Application Data
(60) Provisional application No. 60/972,022, filed on Sep. 13, 2007.

(51) Int. Cl. A61K 35/12; C12N 5/06; A61P 25/00; A61P 9/00; A61P 19/00

(52) U.S. Cl. 424/93.7; 435/378; 435/374; 435/381; 435/377; 435/366

(57) ABSTRACT

A method of isolating a pluripotent cell from human umbilical cord is described herein. The method involves collecting a sample of umbilical cord from fetal tissue obtained at less than 20 weeks of gestation, for example a first trimester umbilical cord. The sample is treated to obtain isolated umbilical cord cells, after which the isolated umbilical cord cells are incubated. Stem cells obtained in this way can be differentiated for use in therapeutic applications.

### CROSS REFERENCE TO OTHER APPLICATIONS

This application claims priority from U.S. Provisional application 60/972,022, filed Sep. 13, 2007, the contents of which are herein incorporated by reference.

### FIELD OF THE INVENTION

The present invention relates generally to human stem cells. More particularly, the present invention relates to a method of isolating and expanding stem cells from first trimester umbilical cords.

### BACKGROUND OF THE INVENTION

Stem cells are unspecialized human or animal cells that can produce mature specialized body cells and at the same time replicate themselves. This ability to differentiate into specialized cells has led to much research into their use for treating such fatal diseases and disorders as Parkinson's and Alzheimer's diseases, spinal cord injury, stroke, burns, heart disease, diabetes, osteoarthritis and rheumatoid arthritis. Stem cells are derived from either embryos or adult tissues. Embryonic stem cells are derived from a blastocyst typically containing 200 to 250 cells. Their use has been hampered by the ethical considerations associated with their isolation.

It has also been reported that mesenchymal-like stem cells can be isolated from the perivascular layer of umbilical cords at birth (HUCPVC), or from blood, bone marrow, skin, and other tissues. These postnatal cells have the ability to self-renew and differentiate to all cell types of mesenchymal lineage. Their use, however, has been hampered by the minimal quantities obtained. Furthermore, adult stem cells have significantly restricted differentiation potential, more DNA damage and shorter life spans as compared with pluripotent stem cells derived from fetal tissue.

U.S. Patent Publication 2005/0148074 A1 (Davies et al.) describes a method of isolating progenitor cells from the Wharton's jelly present in human umbilical cord tissue. However, this method requires tissue to be derived from full-term babies.

It is desirable to provide a method for isolating and expanding stem cells from umbilical cord tissue at a stage earlier than full-term.

### SUMMARY OF THE INVENTION

It is an object of the present invention to obviate or mitigate at least one disadvantage of previous methods for obtaining stem cells.

There is described herein a method for isolating and expanding adult stem cells from first trimester umbilical cords. Advantageously, readily available tissue from first trimester umbilical cords is used to yield relatively large amounts of pluripotent cells. According to one aspect of the present invention there is provided a method of isolating a pluripotent cell from human umbilical, the method comprising: collecting a sample of umbilical cord from fetal tissue obtained at less than 20 weeks of gestation; treating the sample to obtain isolated umbilical cord cells; and incubating the isolated umbilical cord cells.

In an additional aspect, the method can further comprise maintaining the isolated umbilical cord cells, storing (for example, by freezing) the isolated umbilical cord cells, and thawing and restoring the isolated umbilical cord cells to viability is described.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures.

FIG. 1 shows a proliferation profile.

FIG. 2 is a micrograph at 100× magnification.

FIG. 3 is a micrograph; part A is at 200× magnification, part B is at 40× magnification.

FIG. 4 is a micrograph showing immunohistochemical staining.

FIG. 5 is a micrograph showing immunocytochemical staining.

FIG. 6 depicts the results from an RT-PCR assay.

FIG. 7 shows EBs in suspension. (A) Magnification 200×; (B) Magnification 400×.

FIG. 8 illustrates immunocytochemistry to identify enzymatically dispersed EBs expression human embryonic germ layers characterization markers.

FIG. 9 shows expression of differentiation markers in embryo body (EB) by RT-PCR.

FIG. 10 shows HUCPVC differentiation to cardiomyocyte-like cells. (A) Magnification 100×; (B) Magnification 40×.

FIG. 11 shows immunocytochemical detection of mesoderm markers on HUCPVC differentiation into cardiomyocytes.

FIG. 12 shows RT-PCR analysis of first-trimester HUCPV cells expression cardiomyocyte marker genes after in vitro differentiation into cardiomyocytes. (A). Differentiated cells express cTnl (lane 2, 416 bp) and alpha-cardiac actin (lane 6, 418 bp). (B). Differentiated cells express desmin (lane 2, 408 bp) and beta-myosin heavy chain (lane 6, 205 bp).

FIG. 13 shows cell morphology changes after HUCPVC cells were differentiated into nerve-like cells. A: magnification 40×. B and C: magnification 100×. D: magnification 200×.

FIG. 14 shows immunocytochemical detection of ectoderm markers on HUCPVC differentiation into nerve-like cells. (A) MAP-2; (B) MBP; (C) beta-tubulin; (D) nestin.

FIG. 15A (Step 2, Enrichment of Nestin Positive Cells) shows that EBs have attached to the tissue culture dish and have differentiated into pancreatic-like cells. FIG. 15B (Step 3, Differentiation to Insulin-Secreting Pancreatic Islet-like Clusters) shows high density in central pancreatic-like cells.

FIG. 16 shows immunocytochemical staining of HUCPVC-derived islet clusters with pancreatic markers.

FIG. 17 shows that HUCPV cells can differentiate into osteogenic and adipogenic lineages. In FIG. 17A, cells appear polygonal (osteoblasts) under the culture condition of osteogenic differentiation. In FIG. 17B, cells were stained with Alizarin Red S. In FIG. 17C, cells were stained with Oil Red O after cultured with adipogenic complete medium.

### DETAILED DESCRIPTION

Generally, the present invention relates to a method for isolating and expanding stem cells, and more particularly a method wherein the stem cells are derived from first trimester umbilical cords. In accordance with one aspect of the present invention, there is provided a method of isolating a pluripotent cell from human umbilical cord, said method comprising: collecting a sample of umbilical cord from fetal tissue obtained at less than 20 weeks of gestation; treating the sample to obtain isolated umbilical cord cells; and incubating the isolated umbilical cord cells. Additional optional steps may also include: maintaining the isolated umbilical cord cells, freezing the isolated umbilical cord cells, and thawing and restoring the isolated umbilical cord cells to viability.

In one embodiment, umbilical cord samples can be obtained at less than 13 weeks of gestation.

According to one exemplary embodiment, collection of the sample can comprise: collecting fetal placenta tissue by surgical aspiration; and separating the umbilical cords from the fetal placenta tissue. Furthermore, the step of treating the sample can comprise: washing the umbilical cord with PBS; cutting the umbilical cord into pieces; treating the umbilical cord pieces with collagenase to obtain isolated umbilical cord cells; and washing the isolated umbilical cord cells with PBS. Any type of collagenase may be used provided it achieves the effect of separating the cells such as, for example, Type I collagenase at 1 mg/mL.

The incubating step may comprise: suspending the isolated umbilical cord cells in a maintenance medium composed of α-MEM, Fetal Bovine Serum, penicillin-streptomycin, and amphotericin; and maintaining the material under appropriate growth conditions of 37° C., 5% CO₂ and changing the maintenance medium every 3-7 days. Other maintenance medium or growth conditions may be appropriate as long as cell viability or growth is maintained.

In accordance with one embodiment of the present invention, the present method can comprise an optional step of maintaining the isolated umbilical cord cells. Ideally, this step can comprise: washing the isolated umbilical cord cells with PBS; adding trypsin-EDTA; harvesting the isolated umbilical cord cells into a tube containing maintenance medium; separating the isolated umbilical cord cells from the maintenance medium; mixing the isolated umbilical cord cells with new maintenance medium; diluting the new maintenance medium containing the isolated umbilical cord cells with additional maintenance medium to obtain a diluted maintenance medium containing the isolated umbilical cord cells; and maintaining the diluted maintenance medium containing the isolated umbilical cord cells under appropriate growth conditions of 37° C., 5% CO₂ and changing the maintenance medium every 3-7 days. Other maintenance medium or growth conditions may be appropriate as long as cell viability or growth is maintained.

As mentioned above, another optional step in the method according to one aspect of the present invention includes freezing the isolated umbilical cord cells. In one embodiment, this step may comprise: washing the isolated umbilical cord cells with PBS; adding trypsin-EDTA; harvesting the isolated umbilical cord cells into a tube containing maintenance medium; separating the isolated umbilical cord cells from the maintenance medium; cooling the isolated umbilical cord cells to 4° C.; mixing the isolated umbilical cord cells with a freezing medium at 4° C., the freezing medium comprising 80% Fetal Calf Serum and 20% DMSO; transferring the freezing medium containing the isolated umbilical cord cells to vials pre-chilled to −70° C.; storing the vials at −70° C. for 24 h; and storing the vials in liquid nitrogen. Other freezing medium may be appropriate as long as cell viability is maintained.

A further optional step in a method according to one aspect of the present invention includes thawing and restoring the isolated umbilical cord cells. In one embodiment, this step may comprise: warming the vials to 37° C.; separating the isolated umbilical cord cells from the freezing medium; mixing the isolated umbilical cord cells with maintenance medium; maintaining the maintenance medium containing the isolated umbilical cord cells under appropriate growth conditions of 37° C., 5% CO₂, for 24 hours; replacing the maintenance medium with new maintenance medium; and maintaining the new maintenance medium containing the material under appropriate growth conditions, said conditions being 37° C., 5% CO₂ and changing the new maintenance medium every 3-7 days. Other maintenance medium or growth conditions may be appropriate as long as cell viability or growth is restored.

The isolated umbilical cord cells may express one or more transcription factors associated with undifferentiated stem cells. Exemplary transcription factors include OCT-4, SOX-2, or Nanog.

The cells isolated according to the method described may undergo transformation into a differentiated cell such as a neuronal cell, an osteoblast, a chondrocyte, a myocyte, an adipocyte, or a β-pancreatic islet cell.

In accordance with another aspect of the present invention, there is provided a method of obtaining a differentiated cell. In one embodiment, this method involves isolating the cell as described above from human umbilical cord and transforming it using any method acceptable to a person skilled in the art.

Treatment of conditions may be achieved as described herein where the condition requires the function of a damaged cell to be supplanted by a cell obtained according to the method described above. Such conditions may include Parkinson's disease, Alzheimer's disease, spinal cord injury, stroke, burn, heart disease, diabetes, osteoarthritis or rheumatoid arthritis.

Pluripotent cells or differentiated cells can be obtained by methods described herein.

While the first trimester of human gestation can be chronologically identified as the first 13 weeks of gestation, as used herein the term “first trimester” can be extended to further encompass from the 14th week to the 20th week of gestation. A person skilled in the art would recognize that cells isolated up to the 20th week of gestation according to the method described herein may still possess the described features found in those cells isolated in the first 13 weeks of gestation.

The steps described can be further sub-divided. In order to collect samples, fresh fetal-placenta samples are collected from first trimester terminated pregnancies by surgical aspiration into an aseptic bottle; the samples are then moved into dishes where they are searched using forceps, blades and Iris™ scissors for umbilical cords, which are removed.

In one embodiment, treating the umbilical cord sample involves: washing the sample several times with the Dulbecco's Phosphate Buffered Saline (PBS); cutting the sample into small pieces with the curved surgical scissors; transferring the cut sample into a centrifuge tube; digesting the sample; centrifuging; aspirating the supernatant; and washing the sample.

The resulting umbilical cord cells isolated from the treatment of the umbilical cord sample can be incubated by: preparing a warm maintenance medium; re-suspending the isolated umbilical cord cells in the maintenance medium; transferring the isolated umbilical cord cells into tissue culture dishes; adding maintenance medium; and keeping the isolated umbilical cord cells under appropriate growth conditions.

An optional step for the method of isolating cells includes maintaining the isolated umbilical cord cells. The maintenance of isolated umbilical cord cells is undertaken before the cells reach confluence or prior to the growth medium becoming acidic, whichever occurs first. However, the density of cells should exceed approximately 70% of the surface of the culture dishes. In order to maintain the isolated umbilical cord cells, essentially all of the medium is removed from the tissue culture dishes by aspiration; the cells are rinsed once with warmed PBS; room-temperature trypsin-EDTA is added to cover the cells; and the cells are incubated with the trypsin-EDTA until the cells begin to lift off. Cells are harvested into a tube containing maintenance medium and centrifuged to pellet the cell suspension; the media is removed by aspiration and the cells are re-suspended in maintenance medium; and a fraction of the maintenance medium is transferred into a flask containing maintenance medium. Maintenance of the cells in an undifferentiated state can be accomplished by repeating this procedure when the density of the cells exceeds approximately 70% of the surface of the culture flask at each passage.

Another optional step of the method of isolating cells is the storage of the isolated umbilical cord cells. Storage typically involves: freshly preparing freezing medium; harvesting isolated umbilical cord cells using trypsin-EDTA as described above to obtain harvested cells; briefly chilling the harvested cells; re-suspending the harvested cells in ice-cold freezing medium; placing the harvested in pre-chilled cryovials; placing the cryovials in a freezer for 24 hours; and transferring the cryovials to liquid nitrogen for long term storage.

Another optional step of the method of isolating cells is the thawing and restoring of the isolated umbilical cord cells to viability. This step typically involves: warming the maintenance medium; transferring the cryovial in the freezer containing the isolated umbilical cord cells to a water bath; transferring the isolated umbilical cord cells in the cryovial into a centrifuge tube and centrifuging; aspirating the supernatant and re-suspending the isolated umbilical cord cells in an appropriate amount of maintenance medium; and, at a predetermined time after thawing the isolated umbilical cord cells, removing all of the medium and replacing with fresh maintenance medium.

### EXAMPLES

### Example 1

### Materials—Reagents

A non-exhaustive list of possible reagents to use with the method of the invention is provided below. Penicillin-streptomycin liquid containing 5000 U of penicillin and 5000 mg of streptomycin/mL (GIBCO; cat. no. 15070-63), aliquot and store at −20° C. Amphotericin B solution (250 μg/ml, Sigma; cat. no. A-2942), aliquot and store at −20° C. Dulbecco's phosphate-buffered saline (PBS) (+) (GIBCO™; cat. no. 14040-133), store at 4° C. Sterile water, tissue culture grade (GIBCO; cat. no. 15230-162), store at 4° C. Collagenase Type 1 (GIBCO; cat. no. 21985-023), store at 4° C. α-MEM (GIBCO; cat. no. 12571). Defined fetal bovine serum (HyClone, Logan, Utah; cat. no. 30070-03) aliquot and store at −20° C. Trypsin 0.25%/EDTA (GIBCO; cat. no. 25200-056), store at −20° C. DMSO (Sigma™, cat. no. D-5879), store at room temperature. Any additional and acceptable reagents may be used.

### Materials—Equipment

A non-exhaustive list of possible equipment to be used in the method of the invention follows. Watchmakers' forceps (Fine Science Tools Inc., Vancouver, Canada); Iris scissors (Fine Science Tools Inc., cat. no. 14060-09) and dissecting curved surgical scissors (Fischer Scientific; cat. no. 08-935); single edge blades; Pipetmen (2, 10, 100, 200, and 1000 μL); 1-mL individually wrapped serological pipet (BD Biosciences; cat. no. 357522); 5-mL individually wrapped serological pipet (BD Biosciences; cat. no. 357543); 10-mL individually wrapped serological pipet (BD Biosciences; cat. no. 357551); 25-mL individually wrapped serological pipet (BD Biosciences; cat. no. 357525); 15 ml conical centrifuge tubes, high-clarity polypropylene (BD Biosciences; cat. no. 352196); 50 ml conical centrifuge tubes, high-clarity polypropylene (BD Biosciences; cat. no. 352070); 100×20 mm tissue culture dishes (TPP, cat no. 93100); 100×15 mm Petri dishes (Sigma, P5731); 75 cm² tissue culture flask (BD Biosciences; cat. no. 354114); Nalgene freezing box (Nalge Nunc, Rochester, N.Y.; cat. no. 5100-0001); Cryogenic vials (VWR; cat. no. CA66008-284); UV tissue culture enclosure hood (Labconco); 37° C. water bath (VWR); Humidified incubator (Fisher); Inverted microscope with a range of phase contrast objectives (×4, ×10, ×20, and ×40) (Zeiss); liquid nitrogen storage tank; and a tabletop centrifuge. Any additional and acceptable equipment may be used.

### Collection of Samples

Fresh fetal-placenta samples were collected from pregnancies terminated in the first trimester. The samples were surgically aspirated into an aseptic bottle, and immediately transported from operation room (OR) to the research lab for processing. All further steps described were undertaken in sterile conditions using appropriately sterile techniques. Samples were moved into the 100×15 mm petri dish. The samples were carefully searched for the umbilical cord using the forceps, blades and Iris™ scissors. The first trimester umbilical cord is a clear tube-like tissue connected to placental tissue. It is 0.5-2.0 cm in length and contains 2 vessels and one artery. The rest of any of the samples was put back into the bottle, which was filled with 10% formalin and pathology analysis.

### Treatment of the Samples

Isolated umbilical cord was washed several times with PBS. The umbilical cord was cut into small pieces with the curved Iris™ scissors, and transferred into a 15 mL centrifuge tube. The sample was treated for 1 h with collagenase Type 1 (1 mg/ml) while in the 37° C. water bath. The sample was centrifuged at 800 rpm for 15 min at 4° C. to obtain a cell pellet comprising isolated umbilical cord cells and a collagenase supernatant. The collagenase supernatant was removed by aspiration. The cell pellet was washed twice with PBS, centrifuging each time at 800 rpm for 10 min at 4° C. to obtain a washed cell pellet and a PBS supernatant. The PBS supernatant was removed by aspiration.

### Incubating the Isolated Umbilical Cord Cells

Maintenance medium (50 ml) was prepared by mixing 44 ml of α-MEM with 5 ml FBS, 0.5 ml of 100× Penicillin-streptomycin aliquot, and 100×0.5 ml Amphotericin B. The maintenance medium was warmed to 37° C. in the water bath before use. After treating the sample, the isolated umbilical cord cells were re-suspended in 1 mL of maintenance medium to obtain cells. The cells were transferred into 100×20 mm tissue culture dishes and 9 mL of maintenance medium was added. The dishes were placed in the incubator at 37° C. and 5% CO₂ for 3-7 days. The maintenance medium was changed every 2-3 days.

### Maintenance of the Isolated Umbilical Cord Cells

The media was aspirated from the culture dishes and the cultures were rinsed once with PBS warmed to 37° C. in a water bath. Sufficient room temperature trypsin-EDTA was added to cover the cells, approximately 4 mL for a 100 mm dish. The cells were allowed to incubate at 37° C. until the cells just began to lift off. The cells were harvested into a tube containing 4 mL of maintenance medium and centrifuged to pellet the cell suspension (800 rpm for approximately 10 minutes). The media was removed by aspiration and the cells were re-suspended in approximately 2 mL of maintenance medium. Pipetting up and down against the bottom of the tube 4-6 times ensured that the cell pellet was disrupted to a single cell suspension.

In order to perform a 1:4 split of the cells, 0.5 mL of cells were transferred into a 75 cm² flask containing the balance of 10 mL of maintenance medium. The remainder of the cells could be stored or used for experiments. Continued maintenance as required to sustain the cells in an undifferentiated state was accomplished by repeat the above procedure when the density of cells exceeded 70% of the surface of the culture flask at each passage.

### Storage of Material

Fresh freezing medium was prepared by mixing 80% FCS and 20% DMSO. Cryovials were pre-chilled to −70° C. in a Nalgene™ freezing box and cells at a concentration of between 5×10⁵ and 1×10⁶ cells/mL were harvested with trypsin-EDTA as described above. The majority of the media was removed by aspiration and the cells were chilled on ice for 1-2 min. The final cell pellet was re-suspended in ice-cold freezing medium. The cell solution (0.5 mL per cryovial) was transferred into the pre-chilled cryovials in the Nalgene™ freezing box. The Nalgene™ freezing box containing the cryovials was placed in a −70° C. freezer to arrive at frozen cells. Twenty four (24) hours after the cryovials were placed in the freezer, the frozen cells were transferred to liquid nitrogen for long term storage.

### Thawing and Restoring the Material to Viability

Maintenance medium was warmed to 37° C. The cryovial containing the frozen cells was removed from the freezer and thawed quickly in a 37° C. water bath. Once thawed, the cells were transferred into 15 mL conical centrifuge tubes and centrifuged at 800 rpm for 10 minutes. The supernatant was removed by aspiration and the cell pellet was re-suspended in maintenance medium (5 mL for a 60 mm dish or 25 cm² flask; 10 mL for a 100 mm dish). Pipetting gently ensured that the cell pellet was disrupted into a single cell suspension. Twenty four (24) hours after thawing the cells all media was removed and replaced with fresh maintenance medium.

### Results

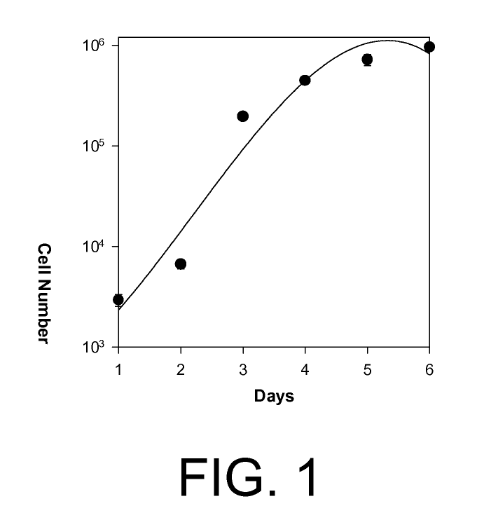

FIG. 1 illustrates the proliferation profile of the cells in the material isolated from first trimester human umbilical cord. The number of cells is plotted against the number of days in culture. The figure shows an increase in cell number over 6 days, indicating that the cells are dividing.

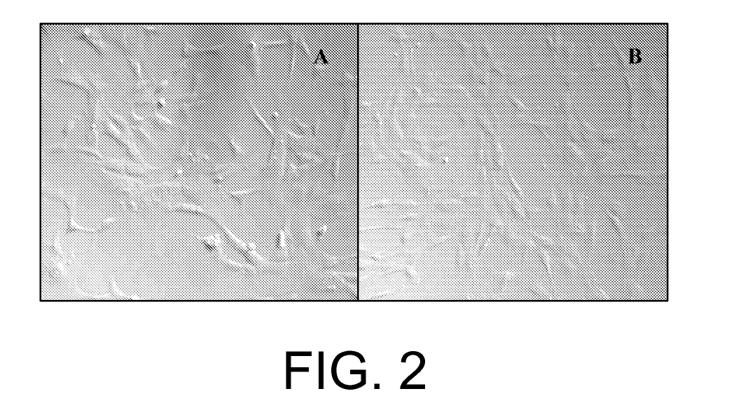

FIG. 2 shows the morphology of the cells in the material isolated from first trimester human umbilical cord. The cells showed homogeneous fibroblast-like morphology. Part A of the figure shows the initial population of the cells at the second passage, while Part B of the figure shows the population of the cells at the 15^{th} passage. In both parts, the magnification is 100×.

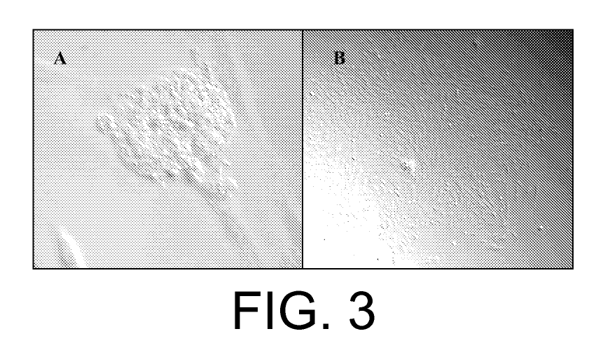

FIG. 3 shows the colony-forming ability of the cells in the material isolated from first trimester human umbilical cord. Cells at earlier passages had a higher frequency of colony-formation as shown in the figure. Part A of the figure shows said cells at the third passage (200× magnification), while Part B of the figure shows said cells at the 7^{th} passage (40× magnification).

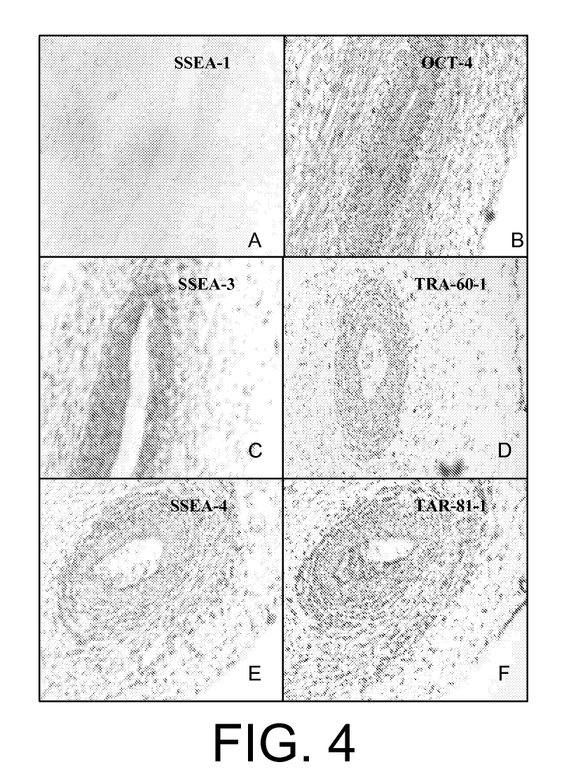

FIG. 4 shows the immunohistochemical (IHC) detection of early embryonic stem cell markers on the cells of first trimester human umbilical cord. IHC analysis revealed that the umbilical cord tissue was positive for TRA-1-60 (FIG. 4D), TRA-1-81 (FIG. 4F), SSEA-3 (FIG. 4C), SSEA-4 (FIG. 4E) and Oct-4 (FIG. 4B), but not SSEA-1 (FIG. 4A). This positive staining was mainly located in the perivascular cell population. This demonstrates that these cells have embryonic stem cell-like properties and derive mainly from the perivascular cell population. This appears to suggest that the markers are characteristic of embryonic stem cells. However, tumor cells that de-differentiate may express some of these markers. They are markers present on embryonic stem cells indicating that they could have the potential for pluripotential differentiation.

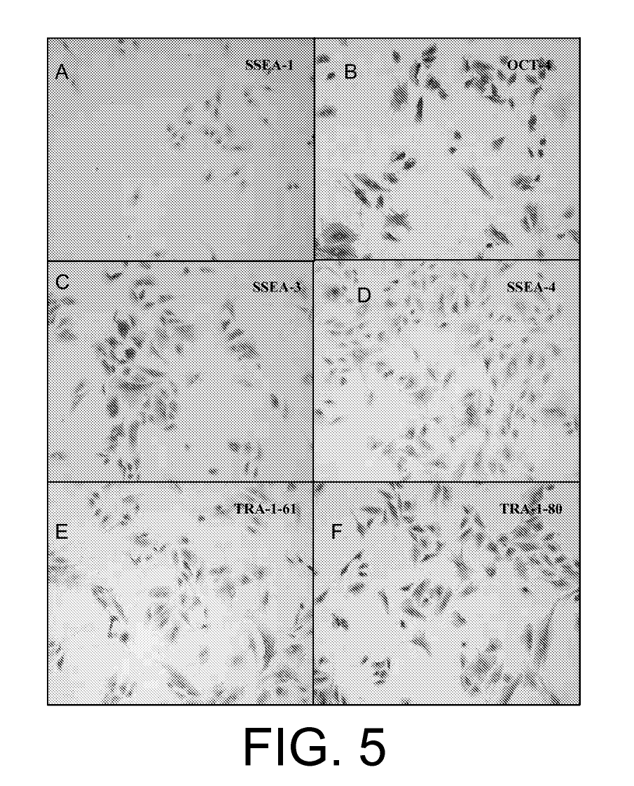

FIG. 5 shows the immunocytochemical detection of early embryonic stem cell markers on the 7^{th} passage of cells in the material isolated from first trimester human umbilical cord. This detection reveals the same markers as those found in the immunohistochemical analysis of the umbilical cord. Expression of these embryonic stem cell markers was consistent from passage 0 to 16, over 11 weeks of culture. Markers: SSEA-1 (FIG. 5A), OCT-4 (FIG. 5B), SSEA-3 (FIG. 5C), SSEA-4 (FIG. 5D), TRA-1-61 (FIG. 5E), and TRA-1-80 (FIG. 5F).

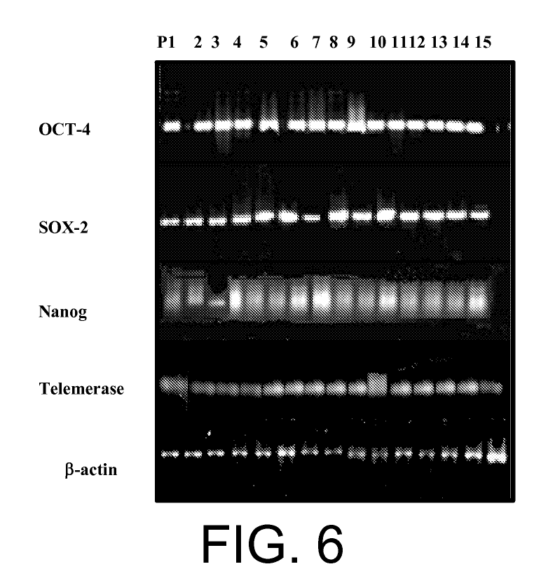

FIG. 6 shows the expression of OCT-4, SOX-2, Nanog and Telemerase transcripts by cells, from passages 1 to 15, in the material isolated from first trimester human umbilical cord. This figure shows that the cells retain expression of these early stem cell markers, and thus retain embryonic stem cell-like properties, for numerous passages in routine culture conditions without showing signs of spontaneous differentiation.

The presence of early embryonic stem markers, along with characteristics such as colony-forming ability, may indicate that material isolated from first trimester umbilical cord cells have embryonic stem cell-like properties. The data provided herein describe immunohistochemical staining that shows the isolated human umbilical cord cells are derives mainly from the perivascular cell population. In addition, the data illustrate the possibility of cryogenic storage and expansion of the isolated human umbilical cord cells, which can be kept undifferentiated in culture. The human umbilical cords isolated from first trimester terminated pregnancies, therefore, have the potential to be a large, and readily obtained source of stem cells. Advantageously, the method described herein does not utilize non-human based feeder layers.

### Example 2

Stem cells were isolated and expanded from the first trimester human umbilical cord (HUCPV), demonstrating that the cells have embryonic stem cell characteristics. These cells can express embryonic stem cell markers from passage 0 to 16 and have the ability to self-renew.

First trimester HUCPV cells were differentiated in vitro and examined for the expression of tissue-specific markers in the differentiated cells.

### Method

Perivascular cells were isolated from first trimester umbilical cords and were expanded in α-MEM containing 5-10% of FCS. These cells were grown in a suspension to induce their differentiation into EBs. EBs were transferred onto coated plates and cultured under appropriate condition. Morphology change was examined in these differentiation conditions. Dispersed EBs and differentiated cells were characterized using immunocytochemistry (ICC). RT-PCR assays were used to detect the presence of several tissue-specific molecular markers.

### Results

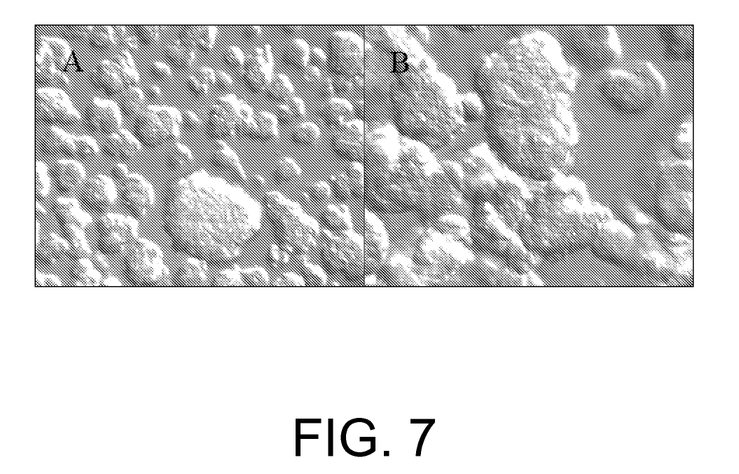

FIG. 7 shows that first trimester HUCPV cells have the ability to form EBs in a suspension culture condition. EBs in suspension may appear individually or as aggregates.

FIG. 7A shows magnification 200×. FIG. 7B shows magnification 400×.

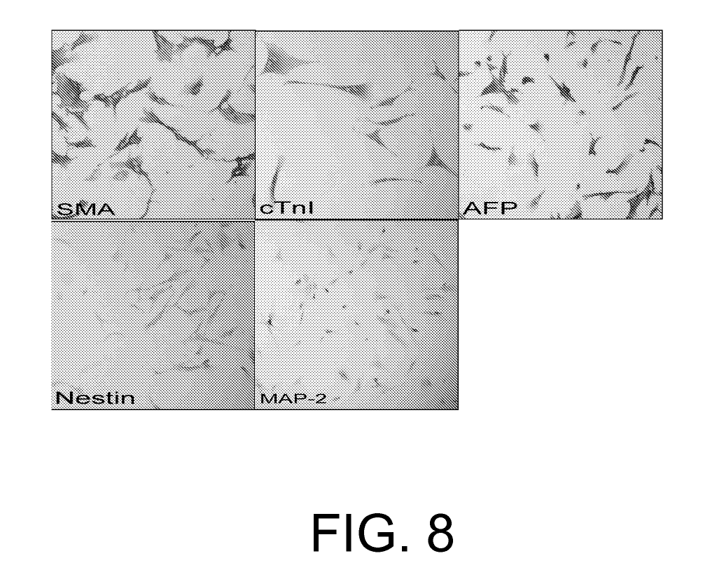

FIG. 8 shows that EBs can express protein markers characteristic of mesoderm (SMA and cTnl), endoderm (AFP), and ectoderm (nestin, MAP-2).

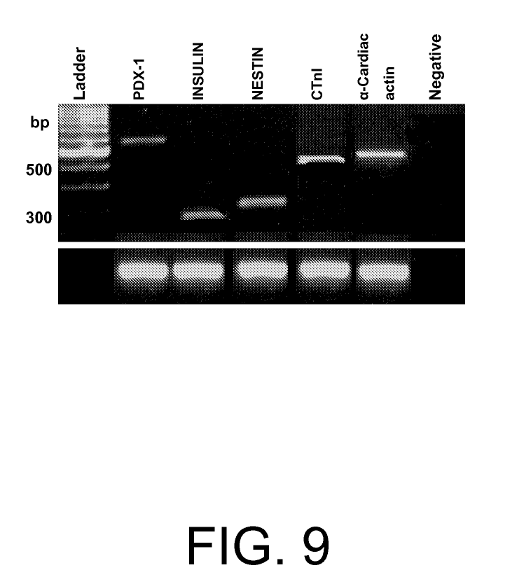

FIG. 9 shows expression of differentiation markers in embryo body (EB) by RT-PCR. This demonstrates the expression of three germ layer markers: PDX-1, insulin, nestin, cTnl and α-cardiac actin.

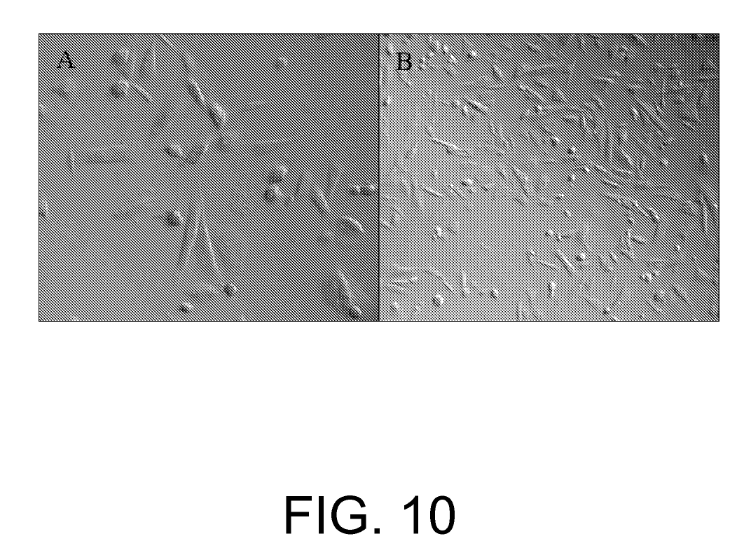

FIG. 10 shows that HUCPV cells have morphology change in cardiomyocytes culture condition. FIG. 10A shows magnification 100×; FIG. 10B shows magnification 40×.

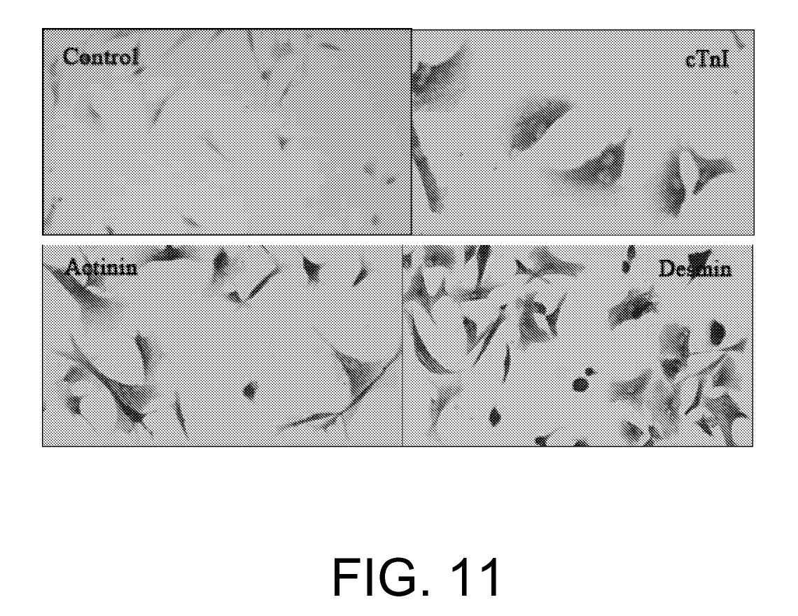

FIG. 11 shows immunocytochemical detection of mesoderm markers on HUCPVC differentiation into cardiomyocytes. Positive immunostaining was identified for cTnl, actin, and desmin.

FIGS. 9 to 11 show that cell morphology changed under differentiation culture conditions.

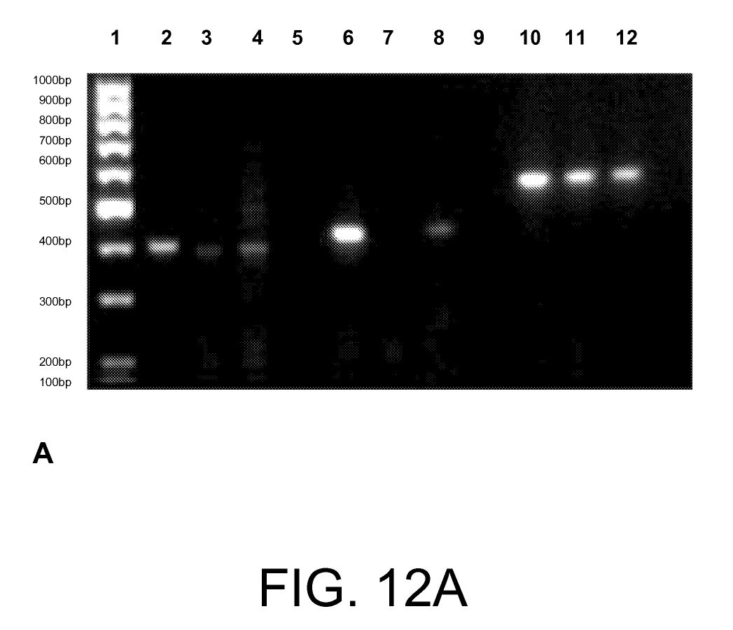

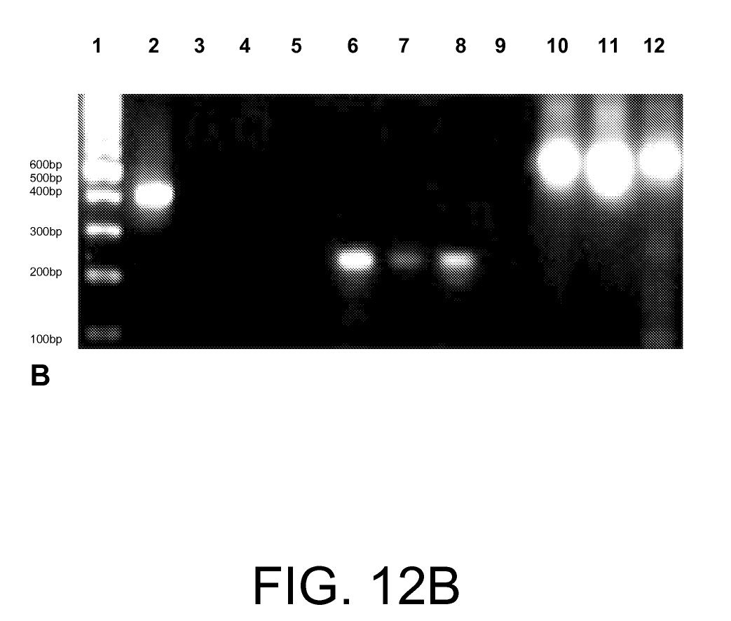

FIG. 12 shows RT-PCR analysis of first-trimester HUCPV cells expression cardiomyocyte marker genes after in vitro differentiation into cardiomyocytes. FIG. 12A shows differentiated cells express cTnl (lane 2, 416 bp) and alpha-cardiac actin (lane 6, 418 bp). FIG. 12B shows that differentiated cells express desmin (lane 2, 408 bp) and beta-myosin heavy chain (lane 6, 205 bp).

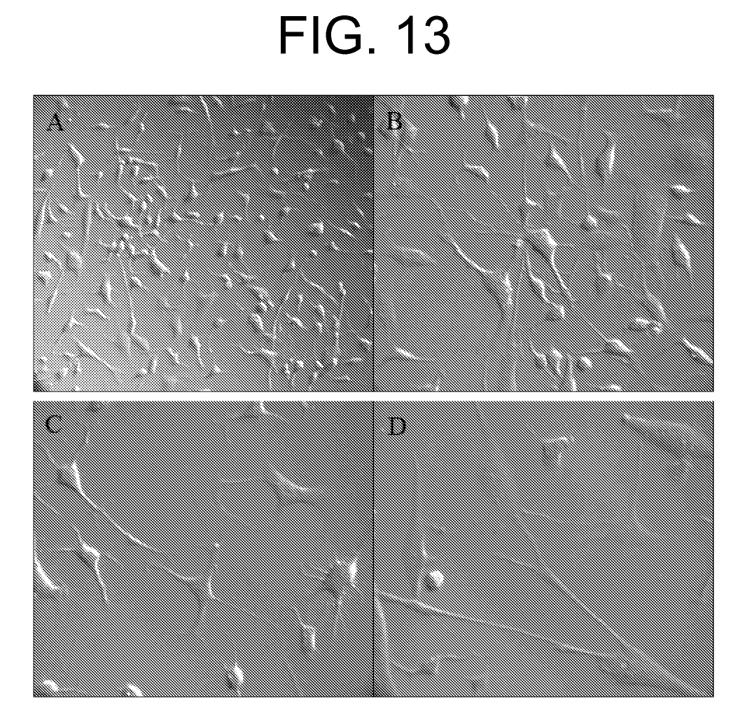

FIG. 13 shows that nerve-like cells can be observed under neural culture conditions. FIG. 13A shows magnification 40×; FIGS. 13B and C show magnification 100×; FIG. 13D shows magnification 200×.

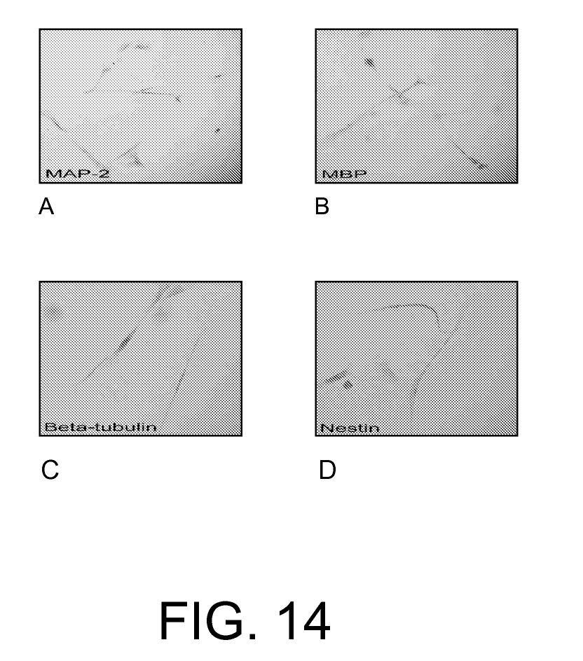

FIG. 14 shows nerve-like cells identified by ICC analysis using neural marker MAP-2, MBP, nestin and β-tubulin. This shows immunocytochemical detection of ectoderm markers on HUCPVC differentiation into nerve-like cells. FIG. 14A shows MAP-2; FIG. 14B shows MBP; FIG. 14C shows beta-tubulin; FIG. 14D shows nestin.

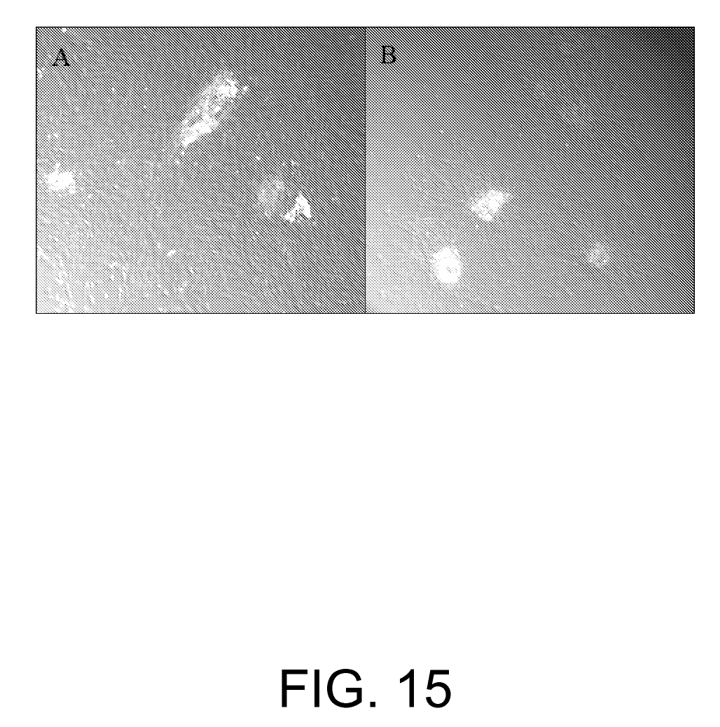

FIG. 15 shows that morphologic changes were observed in pancreatic differentiation stage. In FIG. 15A (Step 2, Enrichment of Nestin Positive Cells), EBs have attached to the tissue culture dish and have differentiated into pancreatic-like cells. In FIG. 15B (Step 3, Differentiation to Insulin-Secreting Pancreatic Islet-like Clusters), high density in central pancreatic-like cells. During this differentiation stage, islets have a three-dimensional topology.

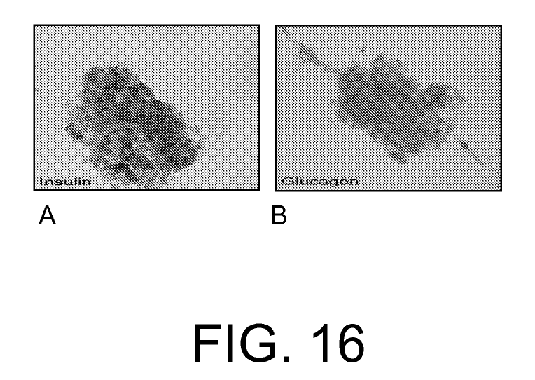

FIG. 16 shows immunocytochemical staining of HUCPVC-derived islet clusters with pancreatic markers. The islet-like clusters can be stained with insulin (FIG. 16A) and glucagon (FIG. 16B).

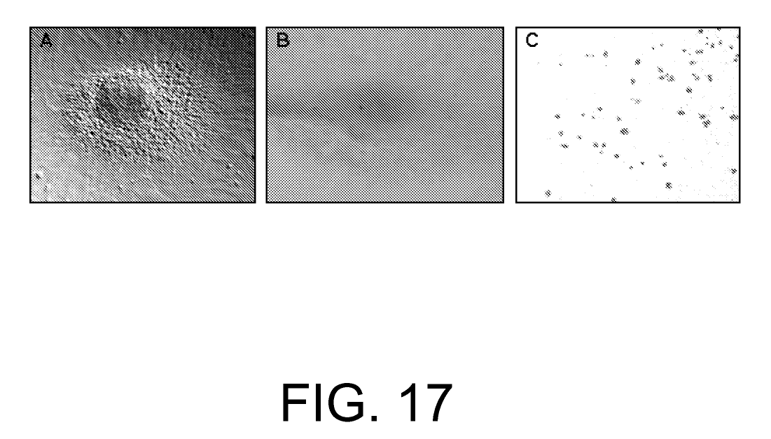

FIG. 17 shows that HUCPV cells can differentiate into osteogenic and adipogenic lineages. These differentiations can be detected by Alizarin Red S and Oil Red O staining. In FIG. 17A, cells appear polygonal (osteoblasts) under the culture condition of osteogenic differentiation. In FIG. 17B, cells were stained with Alizarin Red S. In FIG. 17C, cells were stained with Oil Red O after cultured with adipogenic complete medium.

The above results show that cells derived from human first trimester umbilical cords represent an embryonic-like stem cell population with the capacity to form EBs in vitro. Further, the results show that the cells also have the capacity to differentiate into a wide variety of cell types that include derivatives of all three embryonic germ layers.

In the preceding description, for purposes of explanation, numerous details are set forth in order to provide a thorough understanding of the embodiments of the invention. However, it will be apparent to one skilled in the art that these specific details are not required in order to practice the invention.

The above-described embodiments of the invention are intended to be examples only. Alterations, modifications and variations can be effected to the particular embodiments by those of skill in the art without departing from the scope of the invention, which is defined solely by the claims appended hereto. All documents referred to herein are incorporated by reference.

1. A method of isolating a pluripotent cell from human umbilical cord, said method comprising:
collecting a sample of umbilical cord from fetal tissue obtained at less than 20 weeks of gestation;
treating the sample to obtain isolated umbilical cord cells; and
incubating the isolated umbilical cord cells.

2. A method according to claim 1 additionally comprising:
maintaining the isolated umbilical cord cells;
freezing the isolated umbilical cord cells; and
thawing and restoring the isolated umbilical cord cells to viability.

3. A method according to claim 1 wherein said fetal tissue is obtained at less than 13 weeks of gestation.

4. A method according to claim 1 wherein collecting the sample comprises:
collecting fetal placenta tissue by surgical aspiration; and
separating the umbilical cords from the fetal placenta tissue.

5. A method according to claim 1 wherein treating the sample comprises:
washing the umbilical cord with PBS;
cutting the umbilical cord into pieces;
treating the umbilical cord pieces with collagenase to obtain isolated umbilical cord cells; and
washing the isolated umbilical cord cells with PBS.

6. A method according to claim 5 wherein the collagenase is Type I at 1 mg/mL.

7. A method according to claim 1 wherein incubating the isolated umbilical cord cells comprises:
suspending the isolated umbilical cord cells in a maintenance medium composed of α-MEM, Fetal Bovine Serum, penicillin-streptomycin, and amphotericin; and
maintaining the material under appropriate growth conditions of 37° C., 5% CO₂ and changing the maintenance medium every 3-7 days.

8. A method according to claim 2 wherein maintaining the isolated umbilical cord cells comprises:
washing the isolated umbilical cord cells with PBS;
adding trypsin-EDTA;
harvesting the isolated umbilical cord cells into a tube containing maintenance medium;
separating the isolated umbilical cord cells from the maintenance medium;
mixing the isolated umbilical cord cells with new maintenance medium;
diluting the new maintenance medium containing the isolated umbilical cord cells with additional maintenance medium to obtain a diluted maintenance medium containing the isolated umbilical cord cells; and
maintaining the diluted maintenance medium containing the isolated umbilical cord cells under appropriate growth conditions of 37° C., 5% CO₂ and changing the maintenance medium every 3-7 days.

9. A method according to claim 2 wherein freezing the isolated umbilical cord cells comprises:
washing the isolated umbilical cord cells with PBS;
adding trypsin-EDTA;
harvesting the isolated umbilical cord cells into a tube containing maintenance medium;
separating the isolated umbilical cord cells from the maintenance medium;
cooling the isolated umbilical cord cells to 4° C.;
mixing the isolated umbilical cord cells with a freezing medium at 4° C., said freezing medium comprising 80% Fetal Calf Serum and 20% DMSO;
transferring the freezing medium containing the isolated umbilical cord cells to vials pre-chilled to −70° C.;
storing the vials at −70° C. for 24 h; and
storing the vials in liquid nitrogen.

10. A method according to claim 2 wherein thawing and restoring the isolated umbilical cord cells comprises:
warming the vials to 37° C.;
separating the isolated umbilical cord cells from the freezing medium;
mixing the isolated umbilical cord cells with maintenance medium;
maintaining the maintenance medium containing the isolated umbilical cord cells under appropriate growth conditions of 37° C., 5% CO₂, for 24 hours;
replacing the maintenance medium with new maintenance medium; and
maintaining the new maintenance medium containing the material under appropriate growth conditions, said conditions being 37° C., 5% CO₂ and changing the new maintenance medium every 3-7 days.

11. A method according to claim 1 wherein the isolated umbilical cord cell expresses one or more transcription factor associated with undifferentiated stem cells.

12. A method according to claim 11 wherein the transcription factor is OCT-4, SOX-2, or Nanog.

13. Use of a cell isolated according to claim 1 for transformation into a differentiated cell.

14. The use of a cell according to claim 13 wherein the differentiated cell is a neuronal cell, an osteoblast, a chondrocyte, a myocyte, an adipocyte, or a β-pancreatic islet cell.

15. A method for obtaining a differentiated cell wherein the cell is isolated according to the method of claim 1 and is transformed into a differentiated cell.

16. A method according to claim 15 wherein the differentiated cell is a neuronal cell, an osteoblast, a chondrocyte, a myocyte, an adipocyte, or a β-pancreatic islet cell.

17. A method of treating a condition wherein the function of a damaged cell is supplanted by a cell obtained by the method according to claim 1.

18. A method according to claim 17 wherein the condition is Parkinson's disease, Alzheimer's disease, spinal cord injury, stroke, burn, heart disease, diabetes, osteoarthritis or rheumatoid arthritis.

19. A pluripotent cell obtained by the method of claim 1.

20. A differentiated cell obtained by the method of claim 15.
